(19) United States
(12) Patent Application Publication
(10) Pub. No.: US 2026/0308138 A1
(43) Pub. Date: Oct. 8, 2026

(54) A METHOD AND PLANT FOR PURIFYING ETHYLENE CARBONATE

(71) Applicant: SULZER MANAGEMENT AG, Winterthur (CH)

(72) Inventors: Etienne RIGAUT, Riedisheim (FR); Cecilia MONDELLI, Zürich (CH); Erik TEMMEL, Basel (CH); Yongle LI, Shanghai (CN); Severine DETTE, Winterthur (CH)

(21) Appl. No.: 19/485,547

(22) PCT Filed: May 24, 2024
(86) PCT No.: PCT/EP2024/064358

§ 371 Date: Nov. 18, 2025

(30) Foreign Application Priority Data
May 26, 2023 (WO) PCT/CN2023/096542
Jun. 9, 2023 (EP) 23178428.1

(51) Int. Cl. C07D 317/38; B01D 1/06; B01D 3/14; B01D 3/32; B01D 9/00

(52) U.S. Cl. CPC C07D 317/38; B01D 1/065; B01D 3/141; B01D 3/148; B01D 3/322; B01D 9/004; B01D 9/0059; B01D 2009/0086

(57) ABSTRACT

A method for purifying ethylene carbonate from a crude ethylene carbonate composition includes subjecting a crude composition containing ethylene carbonate to at least one distillation step so as to obtain a pre-purified composition containing at least 99.5% by weight of ethylene carbonate and subjecting the pre-purified composition to a melt crystallization step so as to obtain a purified ethylene carbonate composition. The distillation column is used and is a bottom dividing-wall distillation column including a dividing wall connected with and extending from a bottom of the bottom dividing-wall distillation column upwards over a part of a height of the bottom dividing-wall distillation column thereby subdividing the bottom section seen in cross-section of the dividing-wall distillation column, into a first bottom subsection and into a second bottom subsection.

### CROSS REFERENCE TO RELATED APPLICATION

This application is a U.S. national stage application of International Application No. PCT/EP2024/064358, filed on May 24, 2024 which claims priority to European Patent Application 23178428.1 which was filed on Jun. 9, 2023, and priority to International Application No. PCT/CN2023/096542 which was filed on May 26, 2023, the contents of each of which are hereby incorporated by reference.

### SUMMARY

The present disclosure relates to a method and a plant for purifying ethylene carbonate.

Ethylene carbonate is an important industrial raw material, which is used for instance as solvent or as starting material for the synthesis of ethylene glycols, dimethyl carbonate, ethylene glycol ethers and ethanolamines. Ethylene glycol, in turn, is widely used as chemical, such as for example as antifreeze agent or as monomer in the production of polyester and polyethylene terephthalate, as liquid coolant and as solvent, whereas dimethyl carbonate is used for the manufacture of polycarbonates. Furthermore, ethylene carbonate is gaining more and more importance as electrolyte solvent for lithium-ion batteries, which facilitates the transport of lithium ions from the cathode to the anode. Thereby, ethylene carbonate contributes in a two-fold manner to combat climate change. Firstly, the major route applied in the industrial synthesis of ethylene carbonate is the reaction of ethylene oxide with carbon dioxide, which allows consumption of captured carbon dioxide as a feedstock for green chemicals production, and secondly lithium-ion batteries contribute to the decarbonization of the energy sector through electrification, especially in relation to transport. However, considering that high contents of glycols and, especially, of water are detrimental for the long-term performance of batteries, since they promote the formation of hydrogen fluoride, which in turn affects the quality of the solid electrode interface, ethylene carbonate used as electrolyte solvent for lithium-ion batteries needs to be very pure. Common techniques for purifying crude ethylene carbonate are distillation, crystallization, absorption or the like.

### SUMMARY

It has been discovered however that these methods have at least one of the drawbacks of a low separation efficiency such as but not limited to low depletion factors for glycols and/or water, of a high energy demand and of subjecting the ethylene carbonate to some thermal stress leading to its accelerated degradation and formation of by-products and undesired impurities.

In view thereof, the object underlying the present disclosure is to provide a method and plant for purifying ethylene carbonate being characterized by a particular high productivity, which is the product mass purified per unit of time, by a high recovery yield and by a particular low energy consumption, wherein the method further leads to very pure ethylene carbonate containing 50 ppm or less impurities, such as water and glycols.

In accordance with the present disclosure, this object is satisfied by providing a method for purifying ethylene carbonate from a crude ethylene carbonate composition comprising the steps of:
a) subjecting a crude composition containing ethylene carbonate to at least one distillation step so as to obtain a pre-purified composition containing at least 99.5% by weight of ethylene carbonate and
b) subjecting the pre-purified composition obtained in step a) to at least one melt crystallization step so as to obtain a purified ethylene carbonate composition,
wherein in step a) as the distillation column, if step a) comprises one distillation step, or as the first distillation column, if step a) comprises two or more distillation steps, a bottom dividing-wall distillation column is used, which comprises a dividing wall being connected with and extending from the bottom of the bottom dividing-wall distillation column upwards over a part of the height of the bottom dividing-wall distillation column thereby subdividing the bottom section of the dividing-wall distillation column, seen in cross-section of the dividing-wall distillation column, into a first bottom subsection and into a second bottom subsection.

This solution bases on the finding that by first subjecting a crude ethylene carbonate containing composition, which can for instance originate from a reaction route or can be recovered from any kind of recycling stream, to one distillation step being performed in a bottom dividing-wall distillation column or to two or more distillation steps the first of which being performed in a bottom dividing-wall distillation column, wherein the distillation step(s) lead(s) to a pre-purified composition containing at least 99.5% by weight of ethylene carbonate, before the pre-purified composition is subjected to one or more melt crystallization steps, not only very pure ethylene carbonate containing at most 50 ppm impurities or even not more than 10 ppm impurities, such as water and glycols, is obtained, but also the method can be performed with a particular low energy consumption, nevertheless with a particular high productivity and with a high recovery yield. This is among others due to the fact that the distillation step(s) can be performed under comparable mild conditions, since the crude ethylene carbonate composition is only purified therein to an ethylene carbonate content of at least 99.5% by weight. This does not only allow to keep the ethylene carbonate recovery rate at more than 90%, but also to maintain the energy consumption at a low level, because more stringent distillation conditions or further distillation steps consuming much more energy for achieving a higher purity degree of ethylene carbonate, such as ethylene carbonate containing 200 to 800 ppm impurities, are not necessary. Such a high purity can be achieved by distillation only with an energy consumption being between 3 and 6 times higher compared to the energy consumption of a distillation step being performed under mild conditions so as to obtain a pre-purified composition containing at least 99.5% by weight of ethylene carbonate. In addition, the use of a bottom dividing-wall distillation column significantly reduces the energy consumption of the process even when maintaining the particular high productivity and a high recovery yield. This is due to the fact that the dividing wall subdividing the bottom section of the bottom dividing-wall distillation column, seen in cross-section of the bottom dividing-wall distillation column, into two separate bottom subsections avoids a back-mixing of the bottom fractions between both bottom subsections and hence allows that the bottom fraction being contained in one of the bottom subsections has a higher content of high boiling compounds (meaning compounds having a higher boiling point than ethylene carbonate, such a low or medium molecular weight polymer, catalyst ionic liquid and salt, e.g. calcium and sodium salts) and a lower content of ethylene carbonate than the bottom fraction being contained in the other bottom subsection. This in turn allows increasing the separation efficiency within the distillation column and reducing the energy consumption of the process. In addition, thereby an efficient removal of especially the high boiling compounds from the crude ethylene carbonate composition is achieved, which leads to a reduction of the degradation of ethylene carbonate in the pre-purified composition in the subsequent step(s) and thus to an increase of the ethylene carbonate yield of about 20% during the process. The further purification of the pre-purified composition is then performed in accordance with the present disclosure in one or more melt crystallization steps, which leads to purified ethylene carbonate containing at most 50 ppm or even at most 10 ppm impurities. It has been found in the present disclosure that melt crystallization is characterized by a high separation efficiency for ethylene carbonate from water and glycols for each crystallization stage as well as by very high depletion factors of significantly higher than 10 for glycols, if a pre-purified composition containing at least 99.5% by weight of ethylene carbonate is used as starting composition for the melt crystallization. Depletion factor means with this regard the ratio of the concentration of a substance in the feed of a melt crystallization stage divided by the concentration of the product as obtained as the result of the same melt crystallization stage. Even if it would be possible to purify the crude ethylene carbonate composition solely by melt crystallization without any distillation step, this would require a very high number of crystallization stages in order to obtain a purification to an impurity content of at most 50 ppm. Such a high number of crystallization stages would lead to very high operational costs due to a particular high energy consumption. Furthermore, this would require a large size of the crystallization apparatus. Thus, the distillation step(s) a) leading to just a pre-purified composition containing at least 99.5% by weight of ethylene carbonate and the crystallization step(s) b) synergistically work together and lead with a particular high productivity and with a high recovery yield to a very pure ethylene carbonate containing at most 50 ppm impurities or even not more than 10 ppm impurities at a particular low energy consumption. More specifically, the energy savings are 20 to 25% compared to the known methods.

As set out above, step a) is intentionally performed so as to obtain a pre-purified composition containing at least 99.5% by weight of ethylene carbonate, i.e. a composition, which needs to be not so pure to have only low ppm contents of impurities. Thereby, the number of distillation steps can be kept low as well as the temperature and pressure conditions during the distillation step(s) can be kept moderate so that the energy demand for the distillation is quite low. Good results are in particular obtained, when in step a) at least one distillation step is performed so as to obtain a pre-purified composition containing 99.5 to 99.9% by weight of ethylene carbonate. The content of glycol impurities in the pre-purified composition is preferably 100 to 2,000 ppm. More preferably, in step a) at least one distillation step is performed so as to obtain a pre-purified composition containing 99.6 to 99.9% by weight, even more preferably of 99.7 to 99.9% by weight, still more preferably of 99.8 to 99.9% by weight and most preferably of 99.85 to 99.90% by weight of ethylene carbonate.

In a further development of the idea of the present disclosure, it is suggested that the crude composition used in step a) contains 10 to 99% by weight, preferably 50 to 99% by weight, more preferably 70 to 99% by weight and most preferably 95 to 99% by weight of ethylene carbonate.

As set out above, step a) comprises at least one distillation step. Thus, step a) can comprise two or more distillation steps. However, it is preferred that step a) does not comprise more than two distillation steps. For instance, step a) comprises two subsequent distillation steps. The crude composition is preferably fed in this embodiment in step a) into a first bottom dividing-wall distillation column and is distilled therein into an overhead fraction, a side fraction and a bottom fraction, wherein the bottom fraction of the first bottom dividing-wall distillation column is led into a second distillation column and is distilled therein into an overhead fraction, a side fraction and a bottom fraction, wherein the overhead fraction of the second distillation column is led as the pre-purified composition to the at least one melt crystallization step of step b). However, it is more preferred in this embodiment that the crude composition is fed in step a) into a first bottom dividing-wall distillation column and is distilled therein into an overhead fraction, into a bottom fraction and into a side fraction, wherein the side fraction of the first bottom dividing-wall distillation column is led into a second distillation column and is distilled therein into an overhead fraction, into a bottom fraction and into a side fraction, wherein the side fraction of the second distillation column is led as the pre-purified composition to the at least one melt crystallization step of step b).

In accordance with an alternative particularly preferred embodiment of the present disclosure, step a) comprises (only) one distillation step being performed in a bottom dividing-wall distillation column, wherein the crude composition is fed in step a) into the bottom dividing-wall distillation column and is distilled therein into an overhead fraction, into a bottom fraction and preferably also into a side fraction, wherein preferably the side fraction is led as the pre-purified composition to the at least one melt crystallization step of step b).

Particularly good results are obtained, when the dividing wall extends from the bottom of the bottom dividing-wall distillation column at least substantially vertically upwards over a part of the height of the distillation column thereby subdividing the bottom section of the dividing-wall distillation column, seen in cross-section of the dividing-wall distillation column, into a first bottom subsection and into a second bottom subsection. Bottom section means in accordance with the present disclosure the section of the distillation column extending from the uppermost to the lowermost part of the dividing wall. The first bottom subsection is the subsection of the bottom section being located on the side of the distillation column, into which the inlet for the crude ethylene carbonate composition leads, whereas the second bottom subsection is opposite to the first bottom subsection. Finally, at least substantially vertically means that the angle between the dividing wall and the vertical direction is at most 10°, preferably at most 5°, more preferably at most 1° and most preferably 0°.

In accordance with a further preferred embodiment of the present disclosure, the dividing wall of the bottom dividing-wall distillation column subdivides the bottom section of the bottom dividing-wall dividing-wall distillation column, seen in cross-section of the dividing-wall distillation column, in two equally sized bottom subsections or in two differently sized bottom subsections. Therefore, it is preferred that one of the first and second bottom subsections covers at least 50%, more preferably at least 55%, yet more preferably at least 60% and most preferably 65 to 70% of the total area of the cross-section of the bottom section of the dividing-wall distillation column, whereas the other of the first and second bottom subsections covers the remainder to 100% of the total area of the cross-section of the bottom section of the dividing-wall distillation column. For instance, one bottom subsection covers 43% and the other 57% of the total area of the cross-section or both bottom subsections cover each exactly 50% of the total area of the cross-section. If the total area of the cross-section of the bottom section varies over the axial length of the bottom section, the above numeric values are related to the average total area of the cross-section of the bottom section.

Good results are in particular obtained, when the dividing wall of the bottom dividing-wall distillation column extends in the bottom dividing-wall distillation column from its bottom over 1 to 30% and preferably 5 to 20% of the height of the bottom dividing-wall distillation column.

In accordance with a further particularly preferred embodiment of the present disclosure, the second bottom subsection of the bottom dividing-wall distillation column comprises an outlet line and the bottom dividing-wall distillation column comprises within or above the first bottom subsection an inlet line, both being connected with a recycle line leading from the outlet line to the inlet line, wherein the recycle line is connected with a first reboiler. The first reboiler can comprise two or more heat exchangers in series. The first reboiler comprises one or two vapor outlet lines (or gas outlet lines, respectively) and one liquid outlet line. While the liquid outlet line functions as an outlet line for high boiling compounds via which the liquid fraction with a comparable high content of high boiling compounds is withdrawn from the process, one of the one or two vapor outlet lines is connected with a condenser and downstream thereof with the inlet line leading into the bottom dividing-wall distillation column within or above the first bottom subsection. More specifically, the recycled bottom fraction is partially evaporated in the first reboiler so that a vapor fraction with reduced content of high boiling compounds is obtained and recycled via the above mentioned inlet line into the bottom dividing-wall distillation column, whereas a liquid fraction with an increased content of high boiling compounds is obtained in the first reboiler, which is withdrawn from the process. Good results are in particular obtained, when the first reboiler is a wiped film evaporator, a falling film evaporator or a short path evaporator, more preferably when the first reboiler is a wiped film evaporator or a short path evaporator and most preferably when the first reboiler is a wiped film evaporator.

Likewise thereto, it is also preferred that the first bottom subsection comprises an outlet line and the bottom dividing-wall distillation column comprises within or above the second bottom subsection an inlet line, both being connected with a recycle line leading from the outlet line to the inlet line, wherein the recycle line is connected with a second reboiler. Good results are in particular obtained, when the second reboiler is falling film evaporator.

Preferably, both aforementioned embodiments are combined, i.e. the second bottom subsection comprises an aforementioned recycle line leading from the second bottom sub-section via the first reboiler and its vapor outlet line and subsequent condenser to the first bottom subsection of the bottom dividing-wall distillation column, wherein the first reboiler is preferably a wiped film evaporator, a falling film evaporator or a short path evaporator, and the first bottom subsection comprises an aforementioned recycle line leading from the first bottom subsection via the second reboiler into the second bottom subsection of the bottom dividing-wall distillation column, wherein the second reboiler is preferably a falling film evaporator. It is particularly preferred in this embodiment that the first reboiler, which is more preferably a wiped film evaporator or a short path evaporator, is operated at an operating pressure that is at least 25%, preferably 40% and even more preferably 50% lower than that of the second reboiler. This embodiment allows particularly efficient exploitation of the differences of volatility of the single components of the crude ethylene carbonate composition, thereby efficiently separating low boiling compounds (meaning compounds having a lower boiling point than ethylene carbonate) and high boiling compounds (meaning compounds having a higher boiling point than ethylene carbonate, such a low or medium molecular weight polymer, catalyst ionic liquid and salt, e.g. calcium and sodium salts) from the ethylene carbonate with low energy consumption. Apart from the fact that the dividing wall avoids a back-mixing of the bottom fractions between both bottom subsections of the bottom dividing-wall distillation column, the two aforementioned recycle lines each being connected with a reboiler to allow operation of the first reboiler, which is preferably a wiped film evaporator or a short path evaporator, so that the composition has a very low residence time in the first reboiler of preferably 3 to 15 seconds. The low residence time contributes to a low energy consumption of the process. Moreover, the partial evaporation of the bottom fraction within the first reboiler leads to a reduction of the content of the high boiling compounds in the vapor fraction obtained in the first reboiler and being recycled into the first bottom subsection of the bottom dividing-wall distillation column.

In addition to the aforementioned dividing wall, the bottom dividing-wall distillation column preferably further comprises a partition wall preferably comprising a first portion extending at least essentially vertically downwards and a second portion being connected with an upper part of the first portion of the partition wall and extending to the inner wall of the bottom dividing-wall distillation column, wherein the first portion of the partition wall extends above and/or at least partially within the second bottom subsection of the bottom dividing-wall distillation column. The preferred inlet line of the bottom dividing-wall distillation column being connected with the second reboiler being connected with the outlet line of the first bottom subsection is preferably connected in this embodiment with the wall of the bottom dividing-wall distillation column at a location being below the second portion of the partition wall and between the upper and lower ends of the first portion of the partition wall. The second portion of the partition wall can extend inclined with regard to the horizontal plane, such as inclined by an angle of more than 10° to 40° with regard to the horizontal plane, or can extend at least substantially horizontal, wherein at least substantially horizontal means that the angle between the second portion of the partition wall and the horizontal plane is at most 10°, preferably at most 5°, more preferably at most 1° and most preferably 0°. The second portion of the partition wall is preferably fluid tightly connected with the inner wall of the bottom dividing-wall distillation column, which means that neither descending liquid nor ascending vapor can cross the second portion of the partition wall. The terms dividing wall and partition wall each denote any kind of wall, wherein the “dividing” and “partition” are only used to easily distinguish between the two walls.

In accordance with a further particularly preferred embodiment of the present disclosure, the bottom dividing-wall distillation column comprises a side fraction outlet and further preferably comprises a separation wall. Also the term separation wall denotes any kind of wall, wherein the “separation” is only used to easily distinguish this wall from the aforementioned dividing wall and the aforementioned partition wall. The separation wall comprises a first portion extending at least essentially vertically downwards and a second portion being connected with an upper part of the first portion of the separation wall and extending to the inner wall of the bottom dividing-wall distillation column. While the first portion of the separation wall extends above and/or at least partially within the first bottom subsection of the bottom dividing-wall distillation column, the preferred inlet line of the bottom dividing-wall distillation column being connected with the first reboiler being connected with the outlet line of the second bottom subsection is preferably connected with the wall of the bottom dividing-wall distillation column at a location being below the second portion of the separation wall and between the upper and lower ends of the first portion of the separation wall. Furthermore, it is preferred that the uppermost point of the second portion of the separation wall is located below the side fraction outlet of the bottom dividing-wall distillation column. The second portion of the separation wall can extend inclined with regard to the horizontal plane, such as inclined by an angle of more than 10° to 40° with regard to the horizontal plane, or can extend at least substantially horizontal, wherein at least substantially horizontal means that the angle between the second portion of the separation wall and the horizontal plane is at most 10°, preferably at most 5°, more preferably at most 1° and most preferably 0°. The second portion of the separation wall is preferably fluid tightly connected with the inner wall of the bottom dividing-wall distillation column, which means that neither descending liquid nor ascending vapor can cross the second portion of the separation wall. The side fraction outlet is located between the top and the bottom and is preferably located, seen from the bottom to the top of the bottom dividing-wall distillation column, at a position being located at 40 to 90% and preferably 60 to 90% of the height of the bottom dividing-wall distillation column.

In a further development of the idea of the present disclosure, it is preferred that the overhead of the bottom dividing-wall distillation column comprises an outlet line and an inlet line, both being connected with a recycle line leading from the outlet line to the inlet line, wherein the recycle line is connected with an overhead condenser or cold trap, respectively, which is connected with a liquid line being connected with the recycle line and which is connected with a gas line.

Good results are in particular obtained, when the sidewall of the bottom dividing-wall distillation column comprises an outlet line and an inlet line, both being connected with a recycle line leading from the outlet line to the inlet line, wherein the recycle line is connected with a side condenser, which is connected with a liquid line being connected with the recycle line and which is connected with a gas line, wherein the liquid line leads back into the bottom dividing-wall distillation column.

In order to minimize the energy consumption during the method, it is further pro-posed to perform the distillation step(s) so as to allow heat integration. For instance, the distillation is performed in the at least one distillation step so that the condensation temperature of the overhead fraction is at least 100 ° C, preferably 110 to 150 ° C and more preferably 115 to 125 ° C. This allows to generate in the overhead condenser a heat transfer medium stream with a temperature of at least 80 ° C, preferably of 90 to 130 ° C and more preferably of 95 to 105 ° C. This heat transfer medium can in turn be used to power another step, such as the sweating and/or melting of the ethylene carbonate crystals obtained in the at least one melt crystallization step, as described further below. This allows a reduction in the overall energy consumption by up to 25%, compared to a respective system without implementation of any heat integration.

Furthermore, it is preferred that the distillation column(s) used in step a) comprise(s) one or more internals, such as one or more beds of structured packings, one or more beds of random packings or one or more trays. This leads to a particular efficient mass and heat transfer between the descending liquid phase and the ascending gas phase. More preferably, the distillation column(s) used in step a) comprise(s) one or more beds of structured packings, such as one to five and preferably two to four beds of structured packings. Good results are in particular obtained, when each of the structured packings has a specific surface area 100 to 750 m²/m³ and more preferably of 150 to 500 m²/m³. In the bottom dividing-wall distillation column used in accordance with the present disclosure, the dividing wall can extend so as to touch or even cross a bed of structured packings. However, it is preferred that the dividing wall of the bottom dividing-wall distillation column does not extend so as to touch or even cross a bed of structured packings.

The present disclosure is neither particularly limited concerning the kind of at least one melt crystallization stage nor concerning the number of melt crystallization stages. Good results are in particular obtained, when the at least one melt crystallization step b) comprises at least one melt crystallization stage being selected from the group consisting of falling film crystallization stages, static crystallization stages or suspension crystallization stages. Falling film crystallization means also dynamic crystallization. Each of the aforementioned crystallization techniques can comprise one to five, preferably one to four, more preferably one to three and most preferably one to two melt crystallization stages.

In accordance with a particularly preferred embodiment of the present disclosure, the at least one melt crystallization step b) comprises at least one falling film crystallization stage. During a falling film crystallization stage, melt to be crystallized flows along a cooled surface downwards, such as along the inside of a cooled tube, which allows crystals to grow from the falling film of melt on the inside surface of the tube, which is cooled by a falling film of a cooling agent co-currently flowing on the outside surface of the tube. High and very reproducible transfer rates are achieved on both side of the tube, wherein the resulting shear at the crystal/liquid interface transports impurities rapidly into the bulk of the melt. Preferably, the falling film crystallizer used for the crystallization stage(s) b) contains a plurality of vertical tubes, in which the crystal layers grow as cylindrical shells, a collection vessel beneath the tubes as well as a circulating pump. Before the start of a crystallization stage, the collection vessel is filled with a batch of the melt to be crystallized. The circulating pump is then started to wet the tubes and initiate crystallization at a certain temperature level, while coolant temperature ramping is started. The melt circulation rate is adjusted to a high value compared to the rate of crystal deposition so that conditions of temperature and composition are approximately uniform across the length of the tubes. The temperature is ramped down at a constant rate until the collecting vessel level drops to a preset value. At this point, melt circulation is discontinued.

Good results are in particular obtained, when step b) comprises one to five, preferably one to four, more preferably one to three and most preferably one to two falling film crystallization stages.

Preferably, the pre-purified composition obtained in step a) and subjected in step b) to at least one melt crystallization step is fed into a first of the two to five falling film crystallization stages so as to produce a first ethylene carbonate enriched crystallized fraction and a residue fraction, wherein the first ethylene carbonate enriched crystallized fraction is fed into a second of the two to five falling film crystallization stages, wherein in any of the second and of the optional third to five falling film crystallization stages an ethylene carbonate enriched crystallized fraction and a residue fraction is produced, wherein each of the ethylene carbonate enriched crystallized fractions produced in the second and the optional third to fourth falling film crystallization stages is fed into a downstream ethylene carbonate crystallization stage and each of the residue fractions produced in the second and the optional third to fifth falling film crystallization stages is fed into an upstream falling film crystallization stage, wherein the ethylene carbonate enriched crystallized fraction obtained in the most downstream of the falling film crystallization stages is the purified ethylene carbonate composition.

In addition, it is preferred that the production of an ethylene carbonate enriched crystallized fraction and of a residue fraction in a crystallization stage comprises the steps of removing the remaining liquid from the crystallization stage as residue fraction after termination of the crystallization in the crystallization stage, of melting the crystal layer obtained in the crystallization stage and of withdrawing the obtained crystal melt as ethylene carbonate enriched crystallized fraction from the crystallization stage.

In a further development of the idea of the present disclosure, it is suggested that before melting the crystal layer obtained in the crystallization stage, one or more sweating steps of the crystal layer are carried out so as to obtain one or more sweating fractions and a purified crystal layer, wherein preferably at least a portion of the first sweating fraction obtained thereby is fed to the remaining liquid which has been removed as residue fraction. Sweating is achieved by increasing the temperature of the crystals to a numeric value being just below the melting point of ethylene carbonate, such as 0.1 to 2 ° C below the melting point of ethylene carbonate, in order to liquefy impurities and assist further draining.

Good results are in particular obtained, when at least one and preferably all of the melt crystallization step(s) is/are performed at a temperature of −10 ° C to 70 ° C, preferably at a temperature of −5° C to 65 ° C and more preferably at a temperature of 0 ° C to 60 ° C.

In accordance with a particular preferred embodiment of the present disclosure, the purified ethylene carbonate composition comprises 50 ppm or less and preferably 10 ppm or less impurities.

In a further aspect, the present disclosure relates to a plant for purifying ethylene carbonate from a crude ethylene carbonate composition, wherein the plant comprises at least one distillation column and at least one crystallizer, wherein distillation column, if the plant comprises one distillation column, or the first distillation column, if the plant comprises two or more distillation columns, is a bottom dividing-wall distillation column, which comprises a dividing wall being connected with and extending from the bottom of the bottom dividing-wall distillation column upwards over a part of the height of the bottom dividing-wall distillation column thereby subdividing the bottom section of the dividing-wall distillation column, seen in cross-section of the dividing-wall distillation column, into a first bottom sub-section and into a second bottom subsection, wherein the bottom dividing-wall distillation column further comprises an inlet line for a crude composition containing ethylene carbonate, an overhead outlet line, a bottom outlet line and a side outlet line, wherein at least one crystallizer comprises an inlet line for a pre-purified composition and an outlet line for a purified ethylene carbonate composition, wherein the side outlet line of the bottom dividing-wall distillation column is connected with the inlet line for a pre-purified composition of the at least one crystallizer. That the side outlet line of the bottom dividing-wall distillation column is connected with the inlet line for a pre-purified composition of the at least one crystallizer means a direct or indirect connection of the side outlet line of the bottom dividing-wall distillation column with the inlet line for a pre-purified composition of the at least one crystallizer. An indirect connection means in particular that the side outlet line of the bottom dividing-wall distillation column leads as feed line into a second distillation column comprising an overhead outlet line, a side outlet line and a bottom outlet line, wherein preferably the side outlet line is directly connected with the inlet line for a pre-purified composition of the at least one crystallizer.

Preferably, the dividing wall extends from the bottom of the bottom dividing-wall distillation column at least substantially vertically upwards over a part of the height of the bottom dividing-wall distillation column, wherein at least substantially vertically means that the angle between the at least one dividing wall (16) and the vertical direction is at most 10°, preferably at most 5°, more preferably at most 1° and most preferably 0°.

Good results are in particular obtained, when the dividing wall extends in the distillation column from its bottom over 1 to 30% and preferably 5 to 20% of the height of the distillation column.

Furthermore, it is preferred that the dividing wall subdivides the bottom section of the dividing-wall distillation column, seen in cross-section of the dividing-wall distillation column, into a first bottom subsection covering at least 30%, more preferably at least 40%, yet more preferably at least 45% and most preferably 50% of the total area of the cross-section of the bottom section of the dividing-wall distillation column, whereas the other of the first and second bottom subsections covers the remainder to 100% of the total area of the cross-section of the bottom section of the dividing-wall distillation column.

In accordance with a further particular preferred embodiment of the present disclosure, the bottom dividing-wall distillation column further comprises a partition wall comprising a first portion extending at least essentially vertically downwards and a second portion being connected with an upper part of the first portion of the partition wall and extending to the inner wall of the bottom dividing-wall distillation column, wherein the first portion of the partition wall extends above and/or at least partially within the second bottom subsection of the bottom dividing-wall distillation column.

In accordance with a further particular preferred embodiment of the present disclosure, the bottom dividing-wall distillation column comprises a side fraction outlet and further comprises a separation wall comprising a first portion extending at least essentially vertically downwards and a second portion being connected with an upper part of the first portion of the separation wall and extending to the inner wall of the bottom dividing-wall distillation column, wherein the first portion of the separation wall extends above and/or at least partially within the first bottom subsection of the bottom dividing-wall distillation column.

In a further development of the idea of the present disclosure it is suggested that the second bottom subsection of the bottom dividing-wall distillation column comprises an outlet line and the bottom dividing-wall distillation column comprises within or above the first bottom subsection an inlet line, both being connected with a recycle line leading from the outlet line to the inlet line, wherein the recycle line is connected with a first reboiler. The first reboiler can comprise two or more heat exchangers in series. The first reboiler preferably comprises one or two vapor outlet lines and one liquid outlet line. While the liquid outlet line functions as an outlet line for high boiling compounds via which the liquid fraction with a comparable high content of high boiling compounds is withdrawn from the plant, one of the one or two vapor outlet lines is connected with a condenser and downstream thereof with the inlet line leading into the bottom dividing-wall distillation column within or above the first bottom subsection. If the bottom dividing-wall distillation column comprises a separation wall in the first bottom subsection, it is preferred that the inlet line being connected with the first reboiler being connected with the outlet line of the second bottom subsection is connected with the wall of the bottom dividing-wall distillation column at a location being below the second portion of the separation wall and between the upper and lower ends of the first portion of the separation wall. Good results are in particular obtained in this embodiment, when the first reboiler is a wiped film evaporator, a falling film evaporator or a short path evaporator, more preferably a wiped film evaporator or a short path evaporator and most preferably a wiped film evaporator.

In accordance with a further particular preferred embodiment of the present disclosure, the first bottom subsection of the bottom dividing-wall distillation column comprises an outlet line and the bottom dividing-wall distillation column comprises within or above the second bottom subsection an inlet line, both being connected with a recycle line leading from the outlet line to the inlet line, wherein the recycle line is connected with a second reboiler. If the bottom dividing-wall distillation column comprises a partition wall in the second bottom subsection, it is preferred that the inlet line being connected with the second reboiler being connected with the outlet line of the first bottom subsection is connected with the bottom dividing-wall distillation column at a location being below the second portion of the partition wall and between the upper and lower ends of the first portion of the partition wall, wherein the uppermost point of the second portion of the separation wall is located below the side fraction outlet. It is preferred in this embodiment that the second reboiler is a falling film evaporator.

Good results are in particular obtained, when the at least one crystallizer comprises one to five, preferably one to four, more preferably one to three and most preferably one to two falling film crystallization stages.

### BRIEF DESCRIPTION OF THE DRAWINGS

The disclosure will be explained in more detail hereinafter with reference to the drawings, in which:

FIG. 1 schematically shows a plant for purifying ethylene carbonate from a crude ethylene carbonate composition in accordance with one embodiment of the present disclosure.

FIG. 2 schematically shows a plant for purifying ethylene carbonate from a crude ethylene carbonate composition in accordance with another embodiment of the present disclosure.

FIG. 3 schematically shows the bottom portion of an alternative bottom dividing-wall distillation column, which can be used in a plant shown in FIGS. 1 and 2.

### DETAILED DESCRIPTION

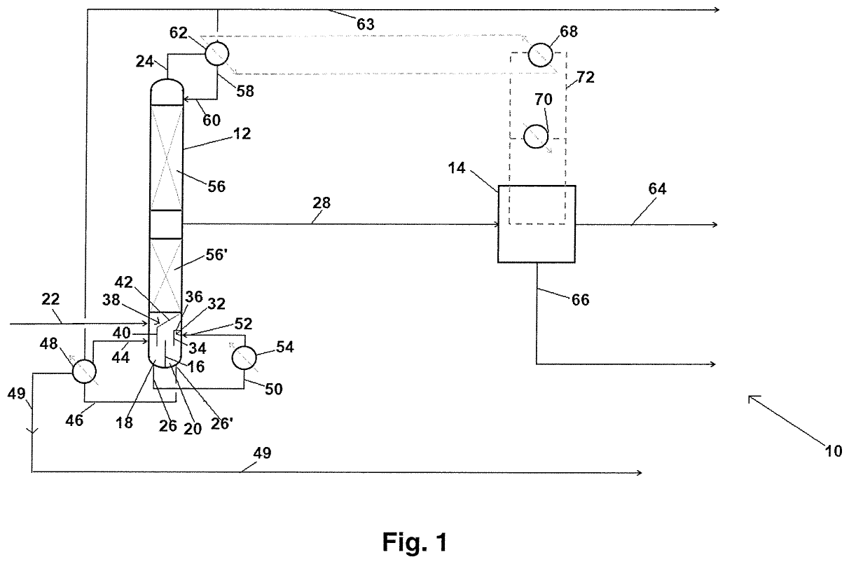

The plant 10 for purifying ethylene carbonate from a crude ethylene carbonate composition shown in FIG. 1 comprises a distillation column 12 and a crystallizer 14. More specifically, the distillation column 12 is a bottom dividing-wall distillation column 12, which comprises a dividing wall 16 being connected with and extending from the bottom of the distillation column 12 vertically upwards over a part of the height of the distillation column 12, thereby subdividing the bottom section of the dividing-wall distillation column 12, seen in cross-section of the dividing-wall distillation column 12, into a first bottom subsection 18 and into a second bottom subsection 20. The dividing wall 16 extends in the distillation column 12 from its bottom over about 10% of the height of the distillation column 12. Moreover, the bottom dividing-wall distillation column 12 comprises an inlet line 22 for a crude composition containing ethylene carbonate, an overhead outlet line 24, two bottom outlet lines 26, 26′ and a side outlet line 28 for pre-purified composition to be led into the crystallizer. In addition, the bottom dividing-wall distillation column 12 comprises a partition wall 32, which comprises a first portion 34 extending vertically downwards and a second portion 36 being connected with the uppermost part of the first portion 34 of the partition wall 32 and extending to the inner wall of the bottom dividing-wall distillation column 12. The first portion 34 of the partition wall 32 extends above and partially within the second bottom subsection 20 of the bottom dividing-wall distillation column 12. Furthermore, the bottom dividing-wall distillation column 12 comprises a separation wall 38, which comprises a first portion 40 extending vertically downwards and a second portion 42 being connected with an upper part of the first portion 40 of the separation wall 38 and extending to the inner wall of the bottom dividing-wall distillation column 12, wherein the first portion 40 of the separation wall 38 extends above and at least partially within the first bottom sub-section 18.

The bottom dividing-wall distillation column 12 further comprises an inlet line 44, which is connected with a recycle line 46 leading from the outlet line 26′ of the second bottom subsection 20 to the inlet line 44, which leads into the first bottom subsection 18 of the bottom dividing-wall distillation column 12. The recycle line 46 is connected with a first reboiler 48, wherein the first reboiler 48 is preferably a wiped film evaporator.

The first reboiler 48 comprises a liquid outlet line 49 for high boiling compounds (heavies), which is in fact a withdrawal line leading out of the plant 10. Furthermore, the first reboiler 48 comprises a vapor outlet line 63 for low boiling compounds (lights) and a vapor outlet line being part of the recycled line 46 and being connected with a condenser (not shown) and downstream thereof with the inlet line 44. Because the first reboiler 48 is usually operated at a lower than the bottom dividing-wall distillation column 12, vapor cannot flow to the sump of the bottom dividing-wall distillation column 12 directly, but is first condensed, before the obtained liquid flows via the inlet line 44 into the bottom dividing-wall distillation column 12 column by gravity. Likewise thereto, also the outlet line 26 of the first bottom subsection 18 is connected with a recycle line 50 leading from the outlet line 26′ to an inlet line 52, which leads into the second bottom subsection 20 of the bottom dividing-wall distillation column 12. The recycle line 50 is connected with a second reboiler 54, wherein the second reboiler 54 is preferably a falling film evaporator. Moreover, the bottom dividing-wall distillation column 12 comprises two beds of structured packing 56, 56′, one 56 of which being arranged above the side outlet line 28 and the other 56′ of which being arranged below the side outlet line 28 and above the separation wall 38. In addition, the outlet line 24 of the overhead of the bottom dividing-wall distillation column 12 is connected with a recycle line 58, which in turn is connected with an inlet line 60 leading into the top portion of the bottom dividing-wall distillation column 12. In turn, the recycle line 58 is connected with an overhead condenser 62, which is in addition connected with an outlet line 63 for lights.

The crystallizer 14 comprises three falling film crystallization stages, an outlet line 64 for pure ethylene carbonate and an outlet line 66 for residue. Furthermore, the crystallizer 14 is connected with a heater 68 and a cooler 70, which form together with the condenser 62 a heat integration system 72.

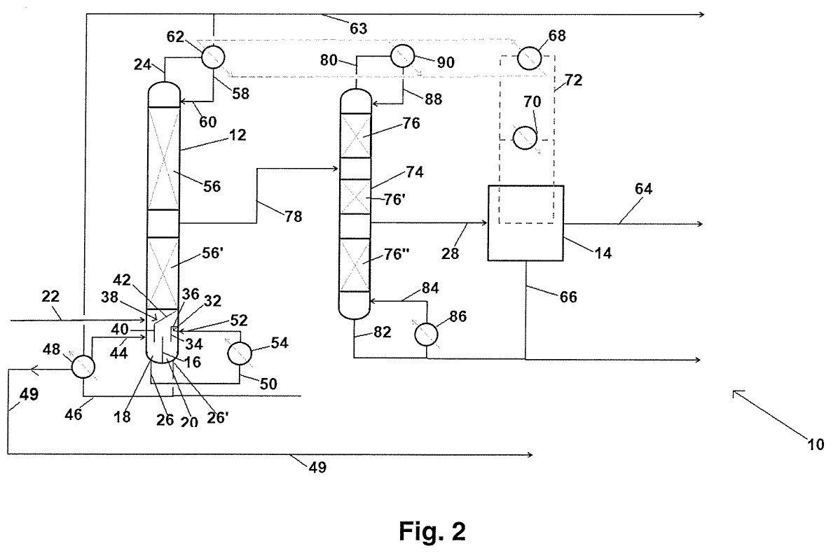

The plant shown in FIG. 2 corresponds to that of FIG. 1 except that it further comprises a second distillation column 74, which is arranged between the bottom dividing-wall distillation column 12 and the crystallizer 14. More specifically, the second distillation column 74 comprises three beds of structured packings 76, 76′, 76″, wherein a connection line 78 leads from the side outlet of the bottom dividing-wall distillation column 12 to the second distillation column 74 and enters the second distillation column 74 at a location between the uppermost and middle beds of structured packings 76, 76′. Also the second distillation column 74 comprises an overhead outlet 80, a bottom outlet 82 and further comprises a recycle line 84 being provided with a reboiler 86 and leading from the bottom outlet line 82 back into the bottom portion of the second distillation column 74. Likewise thereto, the second distillation column 74 further comprises a recycle line 88 being provided with a condenser 90 and leading from the overhead outlet line 80 back into the overhead portion of the second distillation column 74.

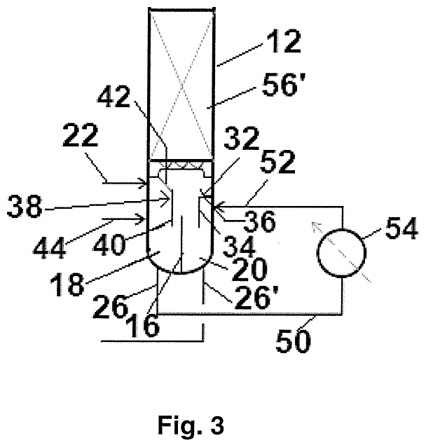

FIG. 3 shows an alternative bottom portion of a bottom dividing-wall distillation column 12, which can be used instead of that shown in FIGS. 1 and 2. The bottom dividing-wall distillation column 12 shown in FIG. 3 corresponds to that shown in FIGS. 1 and 2 except that the shape of the separation wall 38 is slightly different.

1. A method for purifying ethylene carbonate from a crude ethylene carbonate composition, the method comprising:
subjecting a crude composition containing ethylene carbonate to at least one distillation step so as to obtain a pre-purified composition containing at least 99.5% by weight of ethylene carbonate; and
subjecting the pre-purified composition to at least one melt crystallization step so as to obtain a purified ethylene carbonate composition, when subjecting the crude composition containing ethylene carbonate to at least one distillation step, a bottom dividing-wall distillation column is used, the bottom dividing-wall distillation column including a dividing wall connected with and extending from a bottom of the bottom dividing-wall distillation column upwards over a part of a height of the bottom dividing-wall distillation column to subdivide a bottom section of the dividing-wall distillation column, seen in cross-section of the dividing-wall distillation column, into a first bottom subsection and into a second bottom subsection.

2. The method in accordance with claim 1, wherein the bottom dividing-wall distillation column is a first bottom dividing-wall distillation column and the crude composition is fed into the first bottom dividing-wall distillation column and is distilled therein into an overhead fraction, into a bottom fraction and into a side fraction, the side fraction of the first bottom dividing-wall distillation column is led into a second distillation column and is distilled therein into an overhead fraction, into a bottom fraction and into a side fraction, the side fraction of the second distillation column is led as the pre-purified composition to the at least one melt crystallization step.

3. The method in accordance with claim 1, wherein the dividing wall extends from the bottom of the bottom dividing-wall distillation column at least substantially vertically upwards over a part of a height of the bottom dividing-wall distillation column, and at least substantially vertically such that an angle between the at least one dividing wall and a vertical direction is at most 10°.

4. The method in accordance with claim 1, wherein the dividing wall extends in the bottom dividing-wall distillation column from the bottom over 1 to 30% of a height of the bottom dividing-wall distillation column, and, the dividing wall subdivides the bottom section of the bottom dividing-wall distillation column, seen in cross-section of the dividing-wall distillation column, into a first bottom subsection covering at least 50%, of a total area of a cross-section of the bottom section of the bottom dividing-wall distillation column, and a second bottom subsection covering a remainder to 100% of the total area of the cross-section of the bottom section of the dividing-wall distillation column.

5. The method in accordance with claim 4, wherein the second bottom subsection of the bottom dividing-wall distillation column comprises an outlet line and the bottom dividing-wall distillation column comprises within or above the first bottom subsection an inlet line, both the outlet line and the inlet line being connected with a recycle line leading from the outlet line to the inlet line, the recycle line connected with a first reboiler comprising a liquid outlet line and one or two vapor outlet lines one of the one or two vapor outlet lines being connected with a condenser and downstream thereof with the inlet line, and the first bottom subsection comprises an outlet line and the bottom dividing-wall distillation column comprises within or above the second bottom subsection an inlet line, both the outlet line of the first bottom subsection and the inlet line of the bottom dividing-wall distillation column being connected with a recycle line leading from the outlet line of the first bottom subsection to the inlet line of the bottom dividing-wall distillation column the recycle line connected with a second reboiler, the first reboiler being a wiped film evaporator, a falling film evaporator or a short path evaporator and the second reboiler being a falling film evaporator.

6. The method in accordance with claim 1, wherein an overhead of the bottom dividing-wall distillation column comprises an outlet line and an inlet line, both the outlet line and the inlet line of the overhead of the bottom dividing-wall distillation column being connected with a recycle line leading from the outlet line to the inlet line of the overhead of the bottom dividing-wall distillation column, and the recycle line is connected with an overhead condenser, the overhead condenser connected with a liquid line connected with the recycle line which is connected with a gas line.

7. The method in accordance with claim 1, wherein a sidewall of the bottom dividing-wall distillation column comprises an outlet line and an inlet line, both the outlet line and the inlet line connected with a recycle line leading from the outlet line to the inlet line of the sidewall of the bottom dividing-wall distillation column, the recycle line connected with a side condenser, which is connected with a liquid line and which is connected with a gas line, the liquid line leading into the bottom dividing-wall distillation column.

8. The method in accordance with claim 1, wherein the at least one melt crystallization step comprises at least one melt crystallization stage selected from the group consisting of falling film crystallization stage, static crystallization stage and suspension crystallization stage.

9. The method in accordance with claim 1, wherein the subjecting the pre-purified composition to at least one melt comprises one to five falling film crystallization stages.

10. The method in accordance with claim 9, wherein the pre-purified composition obtained and subjected to at least one melt crystallization step is fed into a first of the two to five falling film crystallization stages so as to produce a first ethylene carbonate enriched crystallized fraction and a residue fraction, the first ethylene carbonate enriched crystallized fraction is fed into a second of the two to five falling film crystallization stages, in the second falling film crystallization stage a second ethylene carbonate enriched crystallized fraction and a second residue fraction is produced, the second ethylene carbonate enriched crystallized fraction produced in the second falling film crystallization stage is fed into a downstream ethylene carbonate crystallization stage and the second residue fraction produced in the second falling film crystallization stage is fed into an upstream falling film crystallization stage, the purified ethylene carbonate enriched crystallized fraction is of a most downstream of the falling film crystallization stages.

11. The method in accordance with claim 10, wherein producing the first and second ethylene carbonate enriched crystallized fractions and the first and second residue fractions in a crystallization stage comprises the steps of removing a remaining liquid from the crystallization stage as the first or second residue fraction after termination of the crystallization in the crystallization stage, of melting a crystal layer obtained in the crystallization stage and of withdrawing an obtained crystal melt as the first or second ethylene carbonate enriched crystallized fraction from the crystallization stage, before melting the crystal layer obtained in the crystallization stage, one or more sweating steps of the crystal layer are carried out so as to obtain one or more sweating fractions and a purified crystal layer.

12. The method in accordance with claim 1, wherein the purified ethylene carbonate composition comprises 50 ppm or less impurities.

13. A plant for purifying ethylene carbonate from a crude ethylene carbonate composition, the plant comprising:
at least one distillation column; and
at least one crystallizer,
the distillation column being a bottom dividing-wall distillation column, comprising a dividing wall connected with and extending from a bottom of the bottom dividing-wall distillation column upwards over a part of a height of the bottom dividing-wall distillation column to subdivide a bottom section of the dividing-wall distillation column, seen in cross-section of the dividing-wall distillation column, into a first bottom subsection and into a second bottom subsection, the bottom dividing-wall distillation column further comprising an inlet line for a crude composition containing ethylene carbonate, an overhead outlet line a bottom outlet line and a side outlet line,
the at least one crystallizer comprising an inlet line for a pre-purified composition and an outlet line for purified ethylene carbonate composition, the side outlet line of the bottom dividing-wall distillation column being directly or indirectly connected with the inlet line for pre-purified composition of the at least one crystallizer.

14. The plant in accordance with claim 13, wherein the dividing wall extends from the bottom of the bottom dividing-wall distillation column at least substantially vertically upwards over a part of the height of the bottom dividing-wall distillation column, such that an angle between the at least one dividing wall and the vertical direction is at most 10°

15. The plant in accordance with claim 13, wherein the bottom dividing-wall distillation column further comprises a partition wall comprising a first portion extending at least essentially vertically downwards and a second portion being connected with an upper part of the first portion of the partition wall and extending to an inner wall of the bottom dividing-wall distillation column, the first portion of the partition wall extending above or at least partially within the second bottom subsection of the bottom dividing-wall distillation column.

16. The plant in accordance with claim 13, wherein the bottom dividing-wall distillation column comprises a side fraction outlet and further comprises a separation wall comprising a first portion extending at least essentially vertically downwards and a second portion connected with an upper part of the first portion of the separation wall and extending to an inner wall of the bottom dividing-wall distillation column, the first portion of the separation wall extends above or at least partially within the first bottom subsection of the bottom dividing-wall distillation column.

17. The plant in accordance with claim 16, wherein the second bottom subsection of the bottom dividing-wall distillation column comprises an outlet line and the bottom dividing-wall distillation column comprises within or above the first bottom subsection an inlet line, both the outlet line of the second bottom subsection and the inlet line within or above the first bottom subsection being connected with a recycle line leading from the outlet line of the second bottom subsection to the inlet line within or above the first bottom subsection, the recycle line is connected with a first reboiler comprising a liquid outlet line and one or two vapor outlet lines one of the one or two vapor outlet lines being connected with a condenser and downstream thereof with the inlet line within or above the first bottom subsection, the inlet line within or above the first bottom subsection being connected with the first reboiler being connected with the outlet line of the second bottom subsection is connected with a wall of the bottom dividing-wall distillation column at a location below the second portion of the separation wall and between upper and lower ends of the first portion of the separation wall, the first reboiler is a wiped film evaporator, a falling film evaporator or a short path evaporator.

18. The plant in accordance with claim 17, wherein the first bottom subsection of the bottom dividing-wall distillation column comprises an outlet line and the bottom dividing-wall distillation column comprises within or above the second bottom subsection an inlet line, both the outlet line of the first bottom subsection and the inlet line within or above the second bottom subsection being connected with a recycle line leading from the outlet line of the first bottom subsection to the inlet line within or above the second bottom subsection, the recycle line is connected with a second reboiler, the inlet line within or above the second bottom subsection being connected with the second reboiler being connected with the outlet line of the first bottom subsection is connected with the bottom dividing-wall distillation column at a location being below the second portion of the partition wall of the bottom dividing-wall distillation column and between the upper and lower ends of the first portion of the partition wall, an uppermost point of the second portion of the partition wall is located below the side fraction outlet, the second reboiler is a falling film evaporator.
